(19) United States
(12) Patent Application Publication
(10) Pub. No.: US 2021/0185956 A1
(43) Pub. Date: Jun. 24, 2021

(54) SYSTEM AND METHOD FOR INCREASING CROP YIELDS IN TEMPORARILY INUNDATED FIELDS

(71) Applicant: Jacob L. Martin, Kokomo, IN (US)

(72) Inventor: Jacob L. Martin, Kokomo, IN (US)

(21) Appl. No.: 16/726,240

(22) Filed: Dec. 24, 2019

(51) Int. Cl. A01G 33/00; C05F 11/00; C05G 3/90; A01C 21/00

(52) U.S. Cl. CPC A01G 33/00; A01C 21/00; C05G 3/90; C05F 11/00

(57) ABSTRACT

The present invention is directed as a system method for treating crops with the addition of Cyanobacteria, increasing crops' continuance in the future. According to common knowledge, Cyanobacteria possess a unique set photosynthetic properties, nitrogen-fixing structures, and free radical protection that can be implemented to flora, such as Glycine max. In flooded conditions, crops, on average, die within a time frame of 7 days. Furthermore, this invention offers an approach to culturing Cyanobacteria, such as Oscillatoria or and additional algae.

### CROSS-REFERENCE TO RELATED APPLICATIONS

This application is a continuation of provisional patent application Ser. No. 62/784,659, filed Dec. 24, 2018 entitled, “CROP SURVIVORSHIP UNDER THE CIRCUMSTANCE OF FILED FLOODING WITH THE ADDITION OF CYANOBACTERIA” which is hereby incorporated by reference.

### BACKGROUND OF THE INVENTION

Commercial crops are generally grown in soil which supplies the necessary access to nutrients, moisture, gases, and structural support. The root structure of the plant is the primary interface in the soil.

Since the Agrarian Revolution, dedicated crop land has been used to grow plants for food and fodder. Mechanically, harvested field plant crops are now the primary source of nutrition in humans and domesticated animals across the world. These crops, like all plants, are dependent on oxygen, trace minerals, and carbon dioxide in order to go through the process of photosynthesis.

The use of mechanical farming equipment allowed expansion of growing areas to include non-prime, marginal, and poorly drained areas vulnerable to flooding. In addition, soil compaction and rutting from wheeled or treaded farm machinery can affect drainage even in prime well-drained farmland.

When plant crops are temporarily flooded, (e.g. in low-lying or poorly drained areas of farmland after a heavy rainfall) plant roots are denied access to air for respiration. Once the dissolved oxygen in the flood water is consumed, the water becomes hypoxic and the saturated soil is unable to transfer essential metals and gaseous molecules, resulting in permanent injury to the crop.

Cyanobacteria are a prokaryotic alga, with the ability to photosynthesize and move in the water column, are thought to be responsible for the oxygenic revolution during the Archean time period. Cyanobacteria is capable of surviving in almost all terrestrial environments (e.g. in artic ice, in oceans, in freshwater lakes, rivers, streams, and even in symbiosis with fungus, in high altitude alpine areas).

Cyanobacteria, also known as blue-green algae, have been declared an aquatic pest. Fish and plants have died off from an increase in nutrients through eutrophication, caused by fertilizer runoff. Cyanobacteria, in the bloom, can release a toxin cyanotoxin, when the alga cell dies. Oscillatoria, a genus of blue-green algae, is the primary culprit of these algal blooms, rapidly multiplying and clogging rivers, streams, and ponds.

Oscillatoria has different structures differing from other species of Cyanobacteria, being filamentous. The filamentous Oscillatoria is capable of movement in the water column over a day/night cycle to maximize photosynthesis, but it also uses the filaments to facilitate the exchange of dissolved gases and nutrients.

### SUMMARY OF THE INVENTION

A system and method is presented for mitigating crop losses in temporarily flooded areas using locally collected blue-green algae to increase vital dissolved gases in the flood water. These dissolved gases act to prevent the trapped floodwaters from becoming hypoxic before receding by evaporation or percolation. The blue green algae used can be locally collected due to the worldwide growing range of the algae, preventing the introduction of foreign algae and accidentally collected invasive pests. It is foreseen that governmental boundaries may be set on the transport and use of algae in agriculture to prevent invasive species. One genus of blue-green algae, Oscillatoria, is well suited to the described use, with filamentous growth, innate nitrogen self-fixation, and the production of Butylated Hydroxytoluene (BHT), which acts as an antioxidant. One example of a crop that would be assisted by the described invention is soybeans (Glycine max) which is grown world-wide. In some cases, an algae capable of producing both dissolved oxygen and fixed nitrogen into the flood waters would be cultivated advantageously. Use of customized cultivation systems are described, allowing for the growth of locally-acquired algae and the selection of particular strains that maximize production of the desired factors including oxygen, fixed nitrogen, and BHT. Since the cultivation systems can retain and catalog locally acquired species, a stock of optimized algae varieties can be built up over time.

### BRIEF DESCRIPTION OF THE DRAWING

The foregoing summary as well as the following detailed description is better understood when read in conjunction with the appended drawings. For the purpose of illustrating the invention, there is shown in the drawings exemplary constructions of the invention; however, the invention is not limited to the specific methods and instrumentalities disclosed. In the drawings:

FIG. 1 depicts a representative plant growing in farmland.

FIG. 2 illustrates an exemplarily growth reactor for cultivating algae.

FIG. 3 shows a representative plant in inundated farmland with applied Oscillatoria.

FIG. 4 describes a method for cultivation and application of a local, non-invasive species of Oscillatoria.

### DETAILED DESCRIPTION OF ILLUSTRATIVE EMBODIMENTS

I will now describe illustrative embodiments of the present invention. First, I provide a detailed overview of the problem and then a more detailed description of my solutions.

Flooding is one of the most common natural disasters, creating major destruction. Temporary floods and ponding situations occur commonly due to high amounts of rainfall and inadequate-draining in field locations, creating internal and external damage towards crops, a predominate downfall in a farmer's crop yield.

Short-lived flooding takes over plants roots and prevents the intake of trace minerals and gaseous molecules of nitrogen and oxygen via transport proteins (e.g. leghemoglobin) scattered amongst cell membranes into the cytosol. The plant also drowns from the lack of access to sunlight, preventing photosynthetic metabolism and decreasing transport through the xylem and phloem.

Presently to combat the issue of flooding, farmers in today's world have crop insurance to ensure their crop damages can be compensated. Often times, farmers are forced to replant the crop to replenish their cropland from the damaging flooding or spend tens of thousands of dollars to install a complex drainage system; drainage systems, such as tiling, are unrealistic, as many farmers cannot afford them financially.

With temporary flooding, crop yields are reduced, in some cases to zero. Presented is a system method for using a common, locally sourced blue-green algae to mitigate damage from short-lived flooding of sprouted crops. The common soybean, Glycine max, is used herein as an illustrated example, but other plant crops would benefit from the described invention.

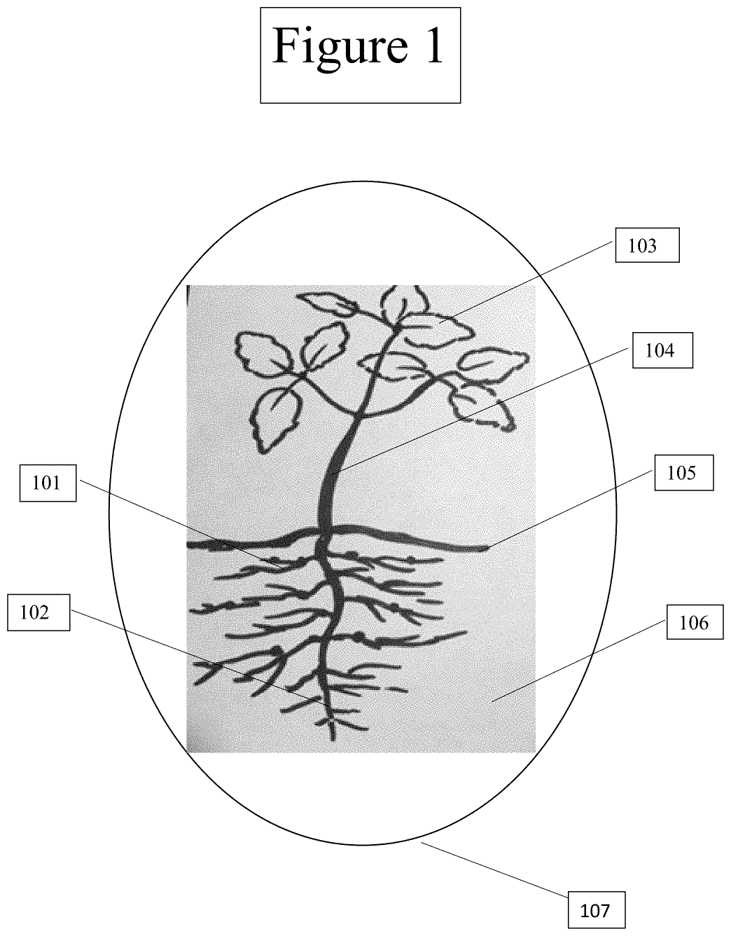

FIG. 1 depicts a representative plant growing in farmland; in this example, a soybean plant with its attendant nitrogen-fixing nodules growing in a normal method.

Nodules 101, present in a soybean plant and other legume crops is a bacteria called Rhizobium; as displayed, the soybean crop contains a type of Rhizobium called Bradyrhizobium japoncium. The nodules have a ball-like appearance onto the diverse root system of a soybean plant. Nodules are very sensitive to oxygen; nodule responses to drought, nitrate, flooding, and photosynthate supply all involve changes in nodule oxygen permeability. Containing the nitrogenase enzyme complex, cellular respiratory process, and leghemoglobin, nodules play a key role in root development inside of the soybean root system. The nitrogenase complex, inside of nitrogen-fixing bacteria, drives the reaction with a large quantity of ATP generated from the nodules own cellular respiratory system involving ATP Synthase, and it uses a collection of metal ions, including an unusual molybdenum ion, to perform the reaction. The protein leghemoglobin is essential to fueling this process. The protein prevents the oxidation of the nitrogenase activity and oxidizes cellular respiration.

Nodules 101, are not normally found in non-leguminous crops. Instead, plant nitrogen needs are supplied by human fertilization or soil bacteria in these other types of plants.

Roots 102, in this example, the roots of a soybean, possess the ability to easily obtain molecules of dissolved substances in the soil through semi-permeable membranes. The root systems in legume crops, with the exemplified soybean, are well-distributed both in and outside the root ball. The roots of the soybean crop are a branching network, thinning out to a string-like structure with a rough surface full of assorted proteins that aid in the transport to the inside of the plant.

Leaf 103, in plants, e.g. the soybean, contains the photosynthesizing mechanism of the plant. The mechanism for photosynthesis requires carbon dioxide from the air via the stoma, and nitrogen, oxygen, and trace minerals collected and transported from the roots.

Stem 104, the stem of the (e.g. soybean) plant is an essential structure. The stem of the plant cannot become flimsy because the plant must be able to obtain an abundance of sunlight. The stem contains the essential passageways of the xylem and phloem. The phloem and xylem serve as the connection point between the root nodules and the photosynthetic thylakoids inside of the chlorophyll of the chloroplast. The vascular xylem is used as the constant source of water and trace metals for the soybean plant, and the phloem is used as the vascular tissue that allows sugars from photosynthesis and other metabolic products to transfer up from the roots and downward from the leaves. The transport between these two vascular structures is different too. The phloem uses a type of process involving sieve plates that can aid in the vertical transport while the xylem is used solely as a undirected one-way transport of water and other minerals.

Soil Line 105, the soil line is where the root system begins below the soil and where the plant stem begins to emerge. The soil line serves as the location for the stem flare. The stem flare is vital for a further developed crop. In crop species not containing a diverse spreading network of roots, these flared stability roots from the stem provide a huge aid in the crops structure, especially in taller plants (such as corn (Zea mays)).

Soil 106, the soil that nourishes crop in has a huge influence on the plant's wellbeing. In soybean species, the soil passes dissolved nitrogen that is essential for fixation. In non-leguminous crops, the soil must contain accessible fixed nitrogen.

Soybean 107 is a crop that produces pods with sets of beans and involves a common sensitivity to flooding. Farmers selectively use this crop due to the cash crop value and its ability to be planted in a wide-range of areas across the entire world. The soybean industry fuels millions of dollars every year from its implications in thousands of everyday items, including foods, oils, and biodiesel fuel.

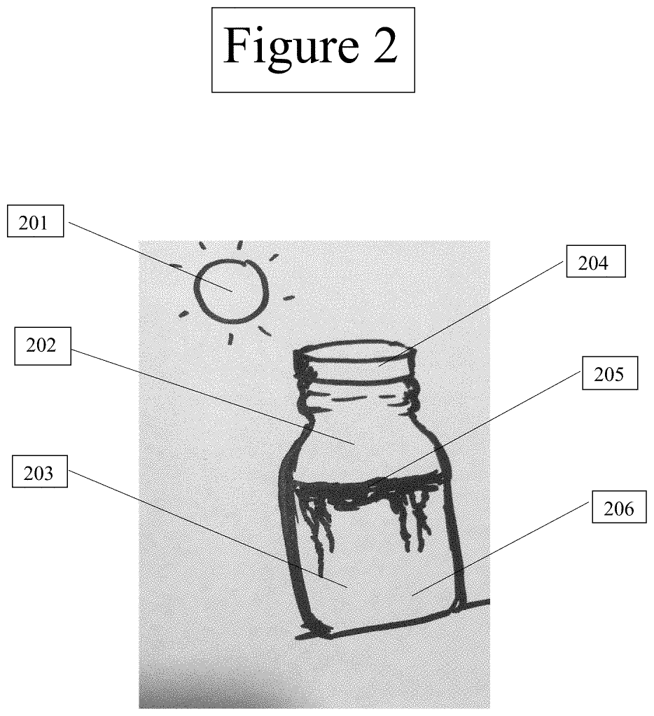

FIG. 2 illustrates an exemplarily growth reactor and method for cultivating algae.

Sun 201, the sun provides sunlight to the soybean leaves for the light dependent reactions of photosynthesis. The sun also provides the light source for the photosynthetic lamellae that undergo the Calvin Cycle in Photosystem II within the Cyanobacteria. In some cases, an artificial illumination method may be used.

Oxygen 202, this gas is produced as a product of photosynthesis, and it is released from the Oscillatoria through the dark reactions with thylakoids. Oxygen is necessary for crop survival because of its link to leghemoglobin, the driving factor for respiration and transportation through the phloem.

Butylated Hydroxytoluene, BHT, 203, an organic compound released from the cyanobacteria, Oscillatoria. BHT is an antioxidant which inhibits oxidation in the protection against free radicals. The BHT has the ability to protect the plants through controlling free radicals that can exist in flooded waters in fields. The BHT prevents inappropriate bonding between atoms.

A container (also known as a reactor) 204, is used for holding and growing Oscillatoria. The container may be transparent for the greater admission of sunlight or may be selectively transparent.

Oscillatoria 205, a type of blue-green algae. The algae release BHT and oxygen. Oscillatoria is also filamentous which allows it to succeed in challenging environments by its string-like structure, creating a large surface area for the absorbance of light during the daytime. Oscillatoria, unlike other Cyanobacteria species, can adhere to crop roots for maximum effectiveness.

The Oscillatoria 205 is advantageously locally collected to prevent introduction of invasive species either of the algae or waterborne organisms. Locality can mean from the farm to be treated or a governmentally-defined ecological (e.g. a watershed or biome) or political area.

Algae-Grow Solution 206, a mixture of water and algae-grow. The solution appears clear in liquid form. The algae-grow solution is a mixture of Ethylenediaminetetraacetic acid (EDTA), Citric Acid, Magnesium Sulfate, Calcium Chloride, and other chemicals depending on the needs of the algae.

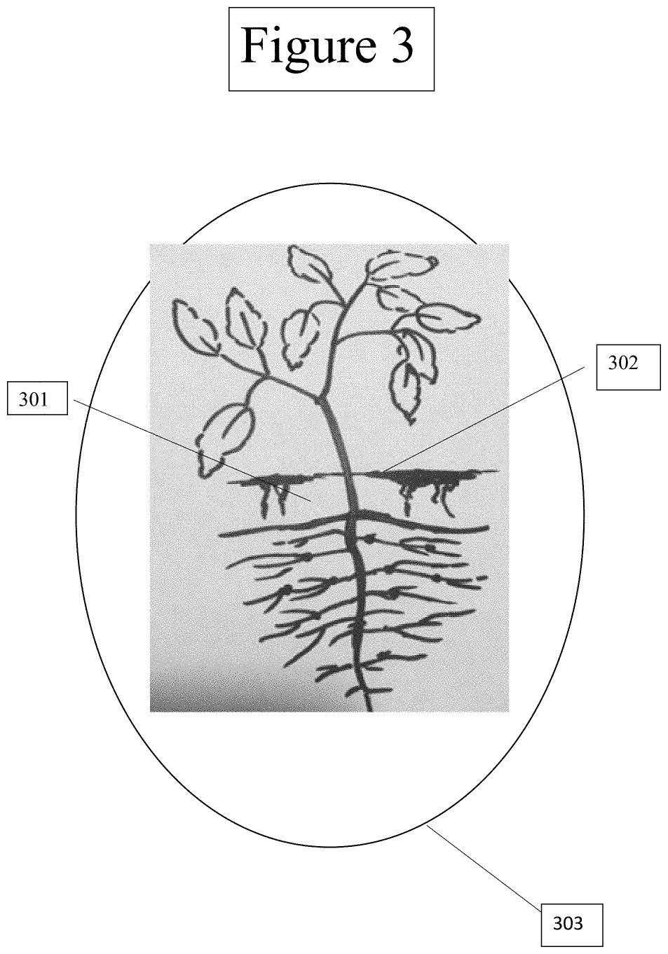

FIG. 3 shows a representative plant in inundated farmland with applied Oscillatoria.

Flooding 301, a natural phenomenon that occurs in areas that have high amounts of rainfall. Flooding can cause traumatic damage to all crops. Flooding interacts differently with every crop. For leguminous crops, floods can prove detrimental after a time period of a week. Flooding saturates the soil, slowing the intake of the soil based nitrogen and trace minerals including molybdenum, magnesium, iron, calcium, and copper. Flooding is considered temporary if it recedes prior to demise of the flooded crop.

Flood Line 302, the highest point of where the flooding occurs. If the flood line is too high, it creates a high amount of weight on soybean roots, increasing stress. This can cause damage to the soybean and defeats the plants access point to an oxygen source. In some cases, the flooding may be deep enough to prevent light from reaching the leaf.

Plant Realm 303, the plant realm is the input and output to the plant. The realm includes all species of compounds, insects, or any disruption that ca occur upon the plant. The plant realm can also include instances of flooding and wind.

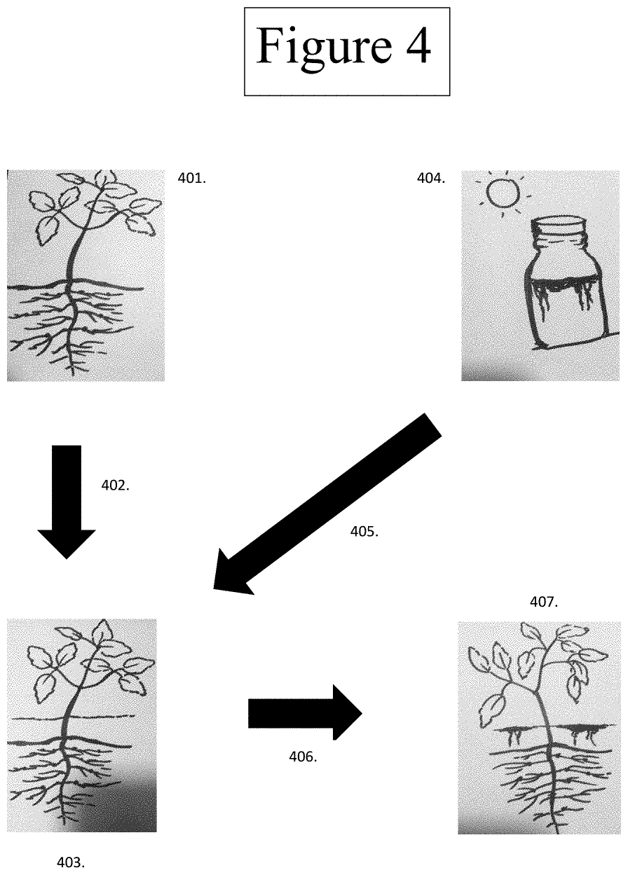

FIG. 4 details the incidence of flooding with the culturing of application of Cyanobacteria e.g. Oscillatoria.

In the first stage 401, a crop plant, such as soybeans, is growing normally in an area susceptible to temporary flooding, e.g. an area with poorly drained soil.

A rainfall event 402, exceeds the drainage capacity of the area of interest resulting in flooding.

In the second stage 403, the plant is inundated, suffocating and losing oxygen accessibility and the ability to take in nitrogen from the soil to thrive in the environment due to the death of the plant.

Separately, in an earlier and parallel stage 404, the collection, culturing, and harvesting of Oscillatoria is performed. This example, depicts an isolated culturing vessel using sunlight for both heating and photosynthesizing in a nutrient rich solution. Both small and large growth reactors are configured based on the size of the local farms and/or the degree of temporary e.g. seasonal field flooding. In large installation, parallel reactors (containers) could be used to cultivate local species that selectively maximize production of dissolved oxygen, dissolved nitrogen, and/or dissolved BHT. A repository of local species can be kept year to year.

In the application stage 405, the cultured Cyanobacteria is applied to the affected. The Cyanobacteria was harvested from the growth reactors and may be applied in a suspended aqueous form or in a dried pelletized form for reconstitution.

After the application of the Cyanobacteria stage 406, the flooded crop is reinvigorated with increase of oxygen and protected with the organic compound BHT within the flooded water.

In the final stage 407, the reinvigorated crop is now cushioned from oxygen deprivation and toxic free radicals. The crop will be demonstratively able to survive the flooding, increasing crop yields and limiting die-off from temporary flooding. Once flooding dissipates, the applied Cyanobacteria dries up but is still available for near-term future cushioning or long-term enriching through decomposition. Since local species of Cyanobacteria were used, disruption of the local ecosystems through invasive Cyanobacteria or unintentionally introduce other species would not be present.

### CONCLUSION

The true scope of the present invention is not limited to the presently preferred embodiments disclosed herein. For example, the foregoing disclosure of a presently preferred embodiment of the system and method for increasing crop yields in temporarily inundated fields uses explanatory terms, such as collect, harvest, sample, and the like, which should not be construed so as to limit the scope of protection of the following claims, or to otherwise imply that the inventive aspects of the system and method for increasing crop yields in temporarily inundated fields are limited to the particular methods and apparatus disclosed. Moreover, as will be understood by those skilled in the art, many of the inventive aspects disclosed herein are based on creative use of generic agricultural machinery and practices. These functional entities (e.g. reactor, sprayers) are, in essence, existing machinery could take a variety of forms without departing from the inventive concepts disclosed herein without changing the inventive operation of the claimed system and methods. Accordingly, except as they may be expressly so limited, the scope of protection of the following claims are not intended to be limited to the specific embodiments described above.

1. A method comprising: collection of a sample of a variety of algae for cultivation, harvesting, and specific application to areas with temporarily inundated planted areas wherein the algae provides dissolved oxygen to mitigate crop losses.

2. The method of claim 1, wherein the said collection of said algae is limited to a geographically local variety.

3. The method of claim 2, wherein the said geographic locality is the same farm.

4. The method of claim 2, wherein the said geographic locality is a governmentally-defined area.

5. The method of claim 1, wherein said algae is a species of the genus Oscillatoria.

6. The method of claim 1 wherein said crop is soybeans.

7. The method of claim 1, wherein the harvested algae has a proven the ability to fix nitrogen.

8. A system for cultivation of collected algae compromising a container, wherein container selectively admits sunlight and air, holds algae growth media, and admits testing and sampling probes.

9. The system as in claim 8, wherein the probes measure dissolved oxygen in the growth media.

10. The system as in claim 8, wherein the probes measure dissolved oxygen and nitrogen in the growth media.

11. The system as in claim 8 wherein the probes measure dissolved Butylated Hydroxytoluene in the growth media.

12. A repository of collected samples of algae wherein the cultivated strains are retained that maximize one or more of the production of dissolved oxygen, fixed nitrogen, or Butylated Hydroxytoluene in the growth media.

13. The repository as in claim 12, wherein the said retained strains are reused in succeeded seasons.

14. The repository as in claim 12, wherein the said retained strains are maintained for separate geographical areas.
